(19) United States
(12) Patent Application Publication
(10) Pub. No.: US 2024/0221959 A1
(43) Pub. Date: Jul. 4, 2024

(54) METHOD AND SYSTEM FOR PREDICTING INDIVIDUALIZED BINARY RESPONSE TO A TREATMENT

(71) Applicant: UCB BIOPHARMA SRL, Brussels (BE)

(72) Inventors: Karim MALKI, Slough Berkshire (GB); Llenalia Maria GERCIA FERNANDEZ, 60 Allee de la Recherche (BE)

(21) Appl. No.: 18/288,612

(22) PCT Filed: Apr. 28, 2022
(86) PCT No.: PCT/EP2022/061361

§ 371 Date: Oct. 27, 2023

(30) Foreign Application Priority Data
Apr. 29, 2021 (EP) EP 21171374.8

(51) Int. Cl. G16H 50/50; G06N 20/20; G16H 30/40; G16H 50/70

(52) U.S. Cl. CPC G16H 50/50; G06N 20/20; G16H 50/70; G16H 30/40

(57) ABSTRACT

A method is provided for constructing a machine learning algorithm for predicting a response variable for a neurological condition. The method includes extracting variables for characterizing a patient cohort from electronic historical medical data; selecting variables from the extracted variables using a random forest defined by an initial response variable; fitting a Bayesian generalized linear mixed model (GLMM) using the initial response variable; extracting a predictive probability from the Bayesian GLMM for each of the selected variables; determining a target response variable from the predictive probability and the initial response variable; using the random forest and the Bayesian GLMM to obtain final estimated predictive probabilities based on target response variable for each of the selected variables to identify relevant selected variables; and constructing the machine learning algorithm to utilize the relevant selected variables for predicting the response variable for the neurological condition.

The present disclosure relates to Artificial Intelligence framework method and system to predict individualized response to a treatment using a patient unique clinical data signature.

### BACKGROUND

There are currently no biomarkers that can be reliably and consistently interrogated to help clinicians drive individualized prescription of treatment medication for neurological conditions such as epilepsy and Parkinson's disease.

Epilepsy is a disease characterized by an enduring predisposition to generate epileptic seizures and by the neurobiological, cognitive, psychological, and social consequences of this condition. A seizure is epileptic if brain electricity monitoring during the event shows unbalanced neurons misfiring. Neuroimaging methods that can measure changes in electrical activation play an important role for the diagnosis of epileptic seizure. Among neuroimaging techniques, EEG is the most common test used to diagnose epilepsy in clinical practice.

EEG is a non-invasive electrophysiological method that continuously records electric potentials and magnetic fields in synchronously-active neurons over a defined window of time. Over the years, improved head models for source estimation have improved both spatial precision of signal detection as well as increased density and coverage. Improvement in computational methods have resulted in sophisticated methods for better signal processing including being able to remove the DC components without allowing the gradients to saturate the input stage and identification and removal of artifacts such as cardiac-related artifacts. This has greatly helped improve the quality of the signal extracted. However, the data generated by EEG technology is high dimensional with many data input available for single observations

The assessment of efficacy in clinical studies evaluating AEDs is generally focused on seizure frequency/occurrence, an approach consistent with regulatory guidelines. Baseline clinical characteristics are routinely collected used as part of clinical assessments, but clinical characteristics have been shown to be poor predictors of response to AED. Univariate analysis of clinical or molecular biomarker have proven unsuccessful in informing individualized response to AED's. The selection of first-prescribed AED's still follows a clinician-based recommendation based on a patient clinical profile including comorbidities, prior or concomitant medications and potential known interactions or a drug side-effect profile. However, multivariable methods of analysis that can learn patterns of statistical regularity across high-dimensional data may hold some promise to find a sparse signal that can predict response to AED treatment in a clinically meaningful way.

Artificial Intelligence (AI) methods have been recently used for detection of epileptiform EEG discharges, as described by Furbass, F., Kural, M. A., Gritsch, G., Hartmann, M., Kluge, T., Beniczky, S. An artificial intelligence-based EEG algorithm for detection of epileptiform EEG discharges: Validation against the diagnostic gold standard. Clinical Neurophysiology, Volume 131, Issue 6, 2020, Pages 1174-1179, ISSN 1388-2457 (https://doi.org/10.1016/j.clinph.2020.02.032). However, to date, there is no method that can use EEG measurements to predict response to pharmacological treatment.

Devinsky et al., “Changing the approach to treatment choice in epilepsy using big data,” Epilepsy & Behavior, Jan. 29, 2016, involves a study utilizing techniques for predict suitable anti-epilepsy drugs (AEDs). This study was only proof of concept, did not provide resources for use in a clinical setting and involved predicting the chances of treatment success, defined by avoidance of hospitalization or treatment change, based on the similarity of the individual patient's characteristics to a larger patient population.

Parkinson's disease is a chronic and progressive movement disorder, such as stiffness, tremor and slowness. There is not cure for the disease, with no disease-modifying pharmacologic treatment, only treatment symptomatic focused on improvement in motor and nonmotor signs are available.

US 2018/0211012 A1 discloses a method of predicting optimal treatment regimens for epilepsy patients, but the method does not at all use neuroimaging data.

### SUMMARY

This present disclosure provides an Artificial Intelligence framework, in the form of a system and method, that uses a combined biological, neuroimaging and clinical signature to predict individualized binary response, such as treatment effect or detect possible adverse events.

The present disclosure provided an AI-based algorithm that combines a ML ensemble method with Bayesian statistical modelling. The AI algorithm is used to select variables without over-fitting the model and solving collinearity issues due to the large number of variables that result from high-throughput data. A Bayesian model is used to model different source of variation (random and systematic variation) providing more robust estimates and further generalization of the solution to the entire target population.

### BRIEF DESCRIPTION OF THE DRAWINGS

The present invention is described below by reference to the following drawings, in which:

FIG. 1 shows a diagram of the method of constructing a machine learning algorithm for predicting the response to a neurological condition;

FIG. 2 shows EEG recordings electrodes;

FIG. 3 shows a system for predicting a response variable for a neurological condition

### DETAILED DESCRIPTION OF THE INVENTION

This present disclosure relates to an artificial intelligence framework that uses a patient biological, neuroimaging, and clinical profile to predict a response variable such as treatment response, need for symptomatic treatment or occurrence of adverse events, among any other possible binary outcome from patients. The identification of a combined biological, physiological and clinical signature can be used for personalized medicine and tailored therapeutics approaches, patient stratification and for the management of diseases.

Within the field of AI, machine learning approaches can be subdivided into supervised and unsupervised methods. Learning is considered supervised when the input and output are known. For example, a supervised algorithm can train on historical patient data with known treatment outcomes. The derived model can then be tested to predict treatment outcome on new patients if presented with the same input data.

Ensemble methods use multiple learning algorithms or the same algorithm multiple times to improve predictive performance. Ensemble methods fall under the class of supervised learning algorithms. They are used to search through a hypothesis space to find a suitable hypothesis that will produce good prediction. For example, a hypothesis in Parkinson disease could be that across all the clinical, neuroimaging and biological variables there is a possible combination of variables that will be associated with treatment outcome patients going on symptomatic treatment. In such cases, it is generally difficult to find a good hypothesis within the hypothesis space that can produce a good prediction. Ensemble methods generate multiple hypothesis using the same base learner, which are then tested within the algorithm. Then, the trained ensemble represents a single hypothesis. Random Forest is an ensemble method where several decision trees are ensembled to take count or average the output from multiple decision trees and return a decision at output. This method provides more stable results because any change in the data set can affect individual decision tree, but it may not affect the whole forest of trees. It also tends to reduce problems of over-fitting of the training data.

Statistical models are a tool that enables the extrapolation from the observed information from a sample to estimate the parameters of a population under study. They are realization of a real problem under study via equations that explain or describe the problem itself. It deals with finding relationship between variable to predict the outcome and quantify the uncertainty. However, since the problem to solve is often influence by external factors that cannot be easily treated, the model or equation needs to incorporate probabilities about the observational data collected. Thus, the probability distributions accommodate for both random and systematic variations. Advice and guidance on how to represent reality by equations and probability functions to solve a problem is required. The art of modelling lies in finding and providing a good technique to describe the real problem and answer the question proposed in the most sensible and possible less complex way.

Bayesian statistical models are statistical models approached from a Bayesian perspective, where the uncertainty is described in terms of random events instead of fixing it with frequencies from repeated measurements under the same condition. The basic principle is that probability is a measure of uncertainty, thus for example the success of treatment is should be treated as random parameter from an unknown distribution of possible values, that can be estimate using different source of information, leading to a potential improvement of the precision of the estimates and predictions.

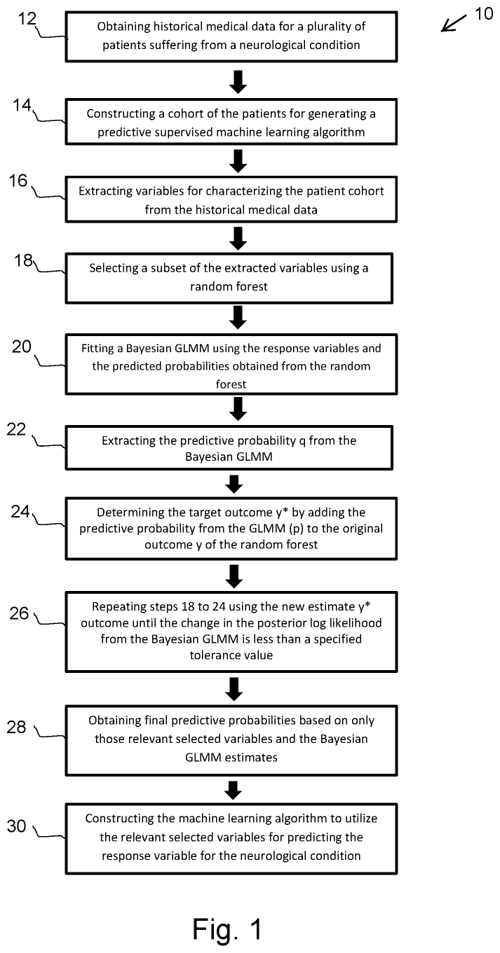

FIG. 1 schematically shows a flow chart of a computational method 10 of generating a predictive supervised machine learning system (algorithm) in accordance with an embodiment of the present disclosure. In particular, method 10 generates an ensemble learning machine learning algorithm. It will be apparent to the skilled person that some or all steps of the method could be implemented using one or multiple computers, e.g. can be performed computationally.

Method 10 may include a first step 12 of obtaining historical medical data for a plurality of patients suffering from a neurological condition.

The provided medical record input data may be subject to pre-processing operations and quality control (QC) checks. Data quality control is performed using statistical tools for outliers and abnormal data structure detection. Where possible, data is corrected for known systematic or user-entered errors. Standardization of the variables is performed at this step. The clean data is then used as input for the iterative methods described below.

Method 10 further includes a step 14 of constructing a cohort of the patients for generating the predictive supervised machine learning algorithm by selecting patients within the historical medical data.

In an example directed to predicting AED success for epilepsy patients, the cohort may be constructed for example in the manner described in US 2018/0211012A1.

In an example directed to predicting symptom treatment for Parkinson's patients, a cohort of Parkinson's patients can be selected. Such cohort may be constructed from a set of patients with a diagnosis of Parkinson Disease (PD) for two or more years without Evidence of a Dopaminergic Deficit. Example of such is available at www.ppmi-info.org/study-design/study-cohorts.

Method further includes a step 16 extracting variables for characterizing the patient cohort from the historical medical data. Neuroimaging, biological and historical-baseline data are extracted and used as main source of extracted variables together with pre-defined clinical variables. Some of pre-defined clinical variables are those that clinicians would consider highly relevant or involved in the disease, such as undertaken treatments or response to it. The historical-baseline data would refer to the history of disease (such as, for example, diabetes, strokes and similar).

The extracted biological variables may include data obtained from laboratory testing of biological samples such a blood, cerebrospinal fluid (CSF), DNA and RNA.

The extracted neuroimaging variables may include MRI or electroencephalography (EEG) data. The extracted clinical variables are those obtained from the clinical history of the patient and their demographic characteristic such as age and sex.

In one example, directed to predicting AED success for epilepsy patients, the extracted variables include neuroimaging, clinical and biological variables.

The clinical variables for epilepsy may include cancer, seizure type (Generalized onset tonic-clonic seizure, simple focal, complex focal, bilateral convulsive seizure, absence, myoclonic, bileteral generalized onset tonic-clonic seizures, non-convulsive, unclear, automatism, auras), indicator for status epilepticus, alcohol, drug abuse, febrile seizures, intracranial bleed, stroke, perinatal damage, infection, head trauma, family history, ethanol withdrawal, photosensitivity, sleep deprivation and proconvulsive medication.

The biological variables for epilepsy may include level of sodium, level of calcium, level of creatine kinase and level of glucose.

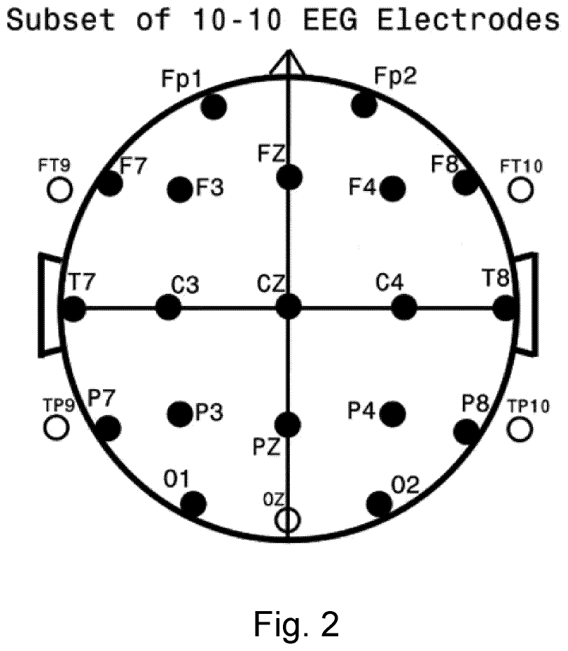

The neuroimaging variables for epilepsy are EEG variables and may include EEG power spectral measurements, which define the decomposition of the signal into functionally distinct frequency bands: Beta (12-30 Hz), Alpha (8-12 Hz), Theta (4-8 Hz), Delta (0.5-4 Hz). FIG. 2 shows a map illustrating the names and positions of EEG electrodes that may be used for performing an EEG for measuring EEG variables in accordance with one preferred embodiment. The Beta, Alpha, Theta and Delta energies, peaks and peak-energies by connectivity area may be used for the AI algorithm. In one example, 216 total EEG variables are used: the 18 connectivity zones (Fp1F3, F3C3, C3P3, P3O1, Fp1F7, F7T7, T7P7, P7O1, FzCz, CzPz, Fp2F4, F4C4, C4P4, P4O2, Fp2F8, F8T8, T8P8, P8O2)×4 EEG frequency bands (Beta, Alpha, Theta, Delta)×3 type of EEG measures (energy, peak, peak energy).

In another example, directed to predicting symptom treatment for Parkinson's patients, the medical record input data may include neuroimaging, clinical and biological variables. These variables may be obtained from Parkinson's progression markers initiative (http://www.ppmi-info.org/about-ppmi/), the goal of which “is to develop disease-modifying treatments that slow, prevent or reserve the underlying disease process.” To achieve the goal, multiple cohorts of patients and clinical sites around the world contribute to defined initiative. A comprehensive set of clinical, neuroimaging and biological data have been designed to help to defined biomarkers of PD progression.

The clinical variables for Parkinson's may include features used to diagnose Parkinson's, including resting tremor present at diagnosis, rigidity present at diagnosis, Bradykinesia present at diagnosis, postural instability present at diagnosis, other symptoms present at diagnosis and the side predominantly affected at onset.

The clinical variables for Parkinson's may include motor variables including Hoehn and Yahr Stage, Modified Schwab and England Capacity for Daily Living percentage score, tremor dominant score, postural instability/gait difficulty, Unified Parkinson's Disease Rating Scale (UPDRS): UPDRS1 (evaluation of mentation, behavior, and mood), UPDRS2 (self-evaluation of the activities of daily life (ADLs)), Sub-UPDRS3—Contralateral, Sub-UPDRS3—Ipsilateral.

The clinical variable for Parkinson's may include non-motor variables including Line Orientation-Sum 15 item X2, Derived-MOANS (Age and Education), Derived-Total Recall T-Score, Derived-Delayed Recall T-Score, Derived-Retention T-Score, Derived-Recog. Discrim. Index T-Score, Derived-LNS Scaled Score, MoCA Total Score, Total Number of animals, Total Number of vegetables, Total Number of fruits, Derived-Sem. Fluency-Animal Scaled Score, Derived-Sem. Fluency-Animal T-Score, Derived-Symbol Digit SD, Derived-Symbol Digit T-Score, Benton judgment of line oriention test (BJLOT), Epworth sleepiness scale (ESS), Geriatric depression scale (GDS), Questionnaire for Impulsive-Compulsive Disorders in PD (QUIP), REM Sleep Behavior disorder (RBD), Scales for outcomes in Parkinson's disease-autonomic (SCOPA-AUT), Semantic fluency, STAI—State Subscore, STAI—Trait Subscore, University of Pennsylvania Smell ID Test (UPSIT), Mild Cognitive Impairment.

The clinical variable for Parkinson's may include vital signs, which may include weight, height, temperature, supine blood pressure (BP)—systolic, supine BP—diastolic, supine heart rate, standing BP—systolic, standing BP—diastolic, and standing heart rate.

The clinical variable for Parkinson's may include variables observed by physical examination, including eyes, cardiovascular (including peripheral vascular), neurological, musculoskeletal, ears/nose/throat, lungs, head/neck/lymphatic, skin, psychiatric and abdomen In general normal vs abnormal variables are compared. Alternatively the variable might be categorical.

The clinical variable for Parkinson's may include variables observed by neurological examination including muscle strength—arm—contralateral, muscle strength—leg—contralateral, coordination—finger-to-nose—contralateral, coordination—heel-to-shin—contralateral, sensory—arm—contralateral, sensory—leg—contralateral, reflex—arm—contralateral, reflex—leg—contralateral, plantar—contralateral, muscle strength—arm—ipsilateral, muscle strength—leg—ipsilateral, coordination—finger-to-nose—ipsilateral, coordination—heel-to-shin—ipsilateral, sensory—arm—ipsilateral, sensory—leg—ipsilateral, reflex—arm—ipsilateral, reflex—leg—ipsilateral, plantar—ipsilateral In general normal vs abnormal variables are compared. Alternatively the variable might be categorical.

The biological variables for Parkinson's may include Albumin-QT (g/L), Alkaline Phosphatase-QT (U/L), ALT (SGPT) (U/L), AST (SGOT) (U/L), Calcium (EDTA) (mmol/L), Creatinine (Rate Blanked) (umol/L), Serum Bicarbonate (mmol/L), Serum Chloride (mmol/L), Serum Glucose (mmol/L), Serum Potassium (mmol/L), Serum Sodium (mmol/L), Serum Uric Acid (umol/L), Total Bilirubin (umol/L), Total Protein (g/L), Urea Nitrogen (mmol/L), APTT-QT (sec), Prothrombin Time (sec), Basophils, Eosinophils, Hematocrit, Hemoglobin, Lymphocytes, Monocytes, Neutrophils, Platelets, RBC, WBC, ABeta 1-42, CSF Alpha-synuclein, pTau, tTau.

The neuroimaging variables for Parkinson's may include the following caudate lobe (CL) and Ipsilateral lobe (IL) MRI imaging variables: CAUDATE_CL, PUTAMEN_CL, CAUDATE_IL, PUTAMEN_IL The putamen is a large structure located within the brain. It is involved in a very complex feedback loop that prepares and aids in movement of the limbs. The caudate nucleus is one of the structures that make up the corpus striatum, which is a component of the basal ganglia. While the caudate nucleus has long been associated with motor processes due to its role in Parkinson's disease, it plays important roles in various other nonmotor functions as well.

Method further includes a step 18 selecting a relevant subset of the variables extracted in step 16 for characterizing the response variable from the patient cohort.

Step 18 involves initializing a random forest by setting a binary response variable, for example an outcome y (yes or no) of whether a neurological treatment will be successful, and predictive variables that are used as predictors of the outcome. The variable selection may be performed in the manner described in Genuer et al., “Variable selection using random forests,” Pattern Recognition Letters, 31(14):2225-2236 (2010). Step 18 also produces predicted probabilities of the response variable y from the random model based on the predictive variables.

Step 18 may include a first substep of computing the random forest scores of importance, then eliminating those variables with importance below a determined threshold and ordering the remaining m variables in decreasing order of importance. The threshold can be selected as the minimum prediction value given by a CART model fitting a curve plotting a standard deviation of importance for each of the remaining variables.

Confounder variables are excluded from the computation at this step. To be a confounder variable, a variable must satisfy the following criteria: (1) it must have an association with the disease, that is, it should be a risk factor for the disease; (2) it must be associated with the exposure, that is, it must be unequally distributed between exposure groups; and (3) it must not be an effect of the exposure. A confounder variable may also not be part of the causal pathway.

Step 18 may then include a second substep of selecting the variables involving the smallest out-of-bag (OOB) error from the random forest varying k variables from k=1 to the total m variables preselected in the first substep of step 18. This finds important variables highly related to the response variable for interpretation in the following steps.

The predicted probabilities of the response from the random model based on the selected variables for interpretation are then used in a next step 20 of method 10, which involves fitting a Bayesian generalized linear mixed model (GLMM) using the response variable, which in this example is whether a neurological treatment will be successful, as the outcome y. The fitting of the Bayesian GLMM includes specifying a fixed effect for the estimated probabilities of success from the random forest with the variables selected in step 18, and a random effect for the subject variability—i.e., whether a neurological treatment will be successful, as well as new inclusion of fixed effects for possible confounder and adjustment variables (for example sex, age, time for longitudinal responses, etc.) In this step, for example, time for the longitudinal response which is not included in the step 16 can be included. Alternatively, or additionally, other variables that could be considered as confounder like sex or age can be included.

For example, steps 20 to 26 may be based on the framework of the Binary Mixed Model (BiMM) forest proposed by Speiser, et al. “BiMM forest: A random forest method for modeling clustered and longitudinal binary outcomes,” Chemometrics and Intelligent Laboratory Systems, Volume 185, 2019, Pages 122-134 (2019).

In the present method the GLMM portion of the BiMM method has the form:

logit(yᵢₜ)=β0+β1*RF(Xᵢₜ)+β2*Confounder1+β3*Confunder2+ . . . +βᵢₜ*Zᵢₜ.

where RF(Xᵢₜ) is represented within the GLMM as the predicted probability from the random forest of each longitudinal observation t1, . . . Tᵢ for cluster i=1, . . . , M. β₀ is the coefficient for the intercept an β₁ is the coefficient for the vector of probabilities; Confounder1 and Confounder 2 are confounder variables and in this case could be age, sex, time of the observed outcome, duration of the disease etc; βᵢₜ is a parameter from the patient random effect; Zᵢₜ is a normal distribution of the random effect. Confounder variables are those obtained from the clinical history of the patient and their demographic characteristics such as age and sex.

More specifically the logit of the probability of initial response variable yᵢₜ to be 1 is calculated using the equation:

$$logit ( {p}_{i t} ) = {β}_{0} + {β}_{1} * RF ( {X}_{i t} ) + {β}_{j 2} {C}_{ij} + {Z}_{i t} {b}_{i t} ,$$

where RF(Xᵢₜ) is the predicted probability from the random forest of each longitudinal observation t1, . . . , Tᵢ for cluster i=1 . . . , M,
β₀ is the coefficient for the intercept,
β₁ is the coefficient for the vector of probabilities,
βᵢₜ is a parameter from the patient random effect,
Zᵢₜ is a normal distribution of the random effect,
βⱼ₂ are the parameters estimated by the model that is adjusted for the confounder variables Cᵢⱼ from the electronic historical medical data,
pᵢₜ the probability of the initial response variable yᵢₜ to be 1, and

$$logit = Ln ( {p}_{it} / ( 1 - {p}_{it} ) )$$

In case of Parkinson Disease, no other variables were included, besides for the confounder disease duration and time when the disease is observed (β₂*Time+β₃*DiseaseDuration+β₄*Age+β₅*Sex), as well as the predictive probability from the random forest (that would correspond to the parameter β₁*RF(Xit)):

$$logit ( {p}_{it} ) = β 0 + β1 * RF ( {X}_{i t} ) + β2 * Time + β3 * DiseaseDuration + β4 * Age + β5 * Sex + {β}_{it} * {Z}_{i t} .$$

Variables, such as PUTAMEN_IL, APTTQT_sec, UPDRS2, PUTAMEN_CL, UPDRS1 are used to estimate the values RF(Xᵢₜ).

In case of epilepsy the clinical information could be: cancer, seizure type (generalized onset tonic-clonic seizure, simple focal, complex focal, bilateral convulsive seizure, absence, myoclonic, bileteral generalized onset tonic-clonic seizures, Non-convulsive, unclear, automatism, auras), level of sodium, level of calcium, level of ck, level of glucose, indicator for status epilepticus, alcohol, drug abuse, febrile seizures, intracranial bleed, stroke, perinatal damage, infection, head trauma, family history, ethanol withdrawal, photosensitivity, sleep deprivation and proconvulsive medication. All those variables could be used in the formulae above, instead of Time and disease duration.

The method 10 further includes a step 22 of extracting the predictive probability q from the Bayesian GLMM for each of the measurements of the outcome y.

Then, method 10 is iterated through steps 18-24 until reaching convergence. Step 24 includes determine the target outcome y* by adding the predictive probability determined in step 22 from the GLMM (p) to the original outcome y of the random forest, and applying a split function y*=h(p+y) to make a binary value (h=1 ify+p>1; 0 otherwise, with 0<k<1). Step 26 includes repeating steps 18 to 24 using the new estimate y* outcome until the change in the posterior log likelihood from the Bayesian GLMM is less than a specified tolerance value.

Method 10 next includes a step 28 to obtain final estimated predictive probabilities based on only those relevant selected variables and the Bayesian GLMM estimates.

Method 10 then includes a step 30 of constructing the machine learning algorithm to utilize the relevant selected variables for predicting the response variable for the neurological condition. From the Bayesian GLMM estimates, the machine learning algorithm determines the response success of treatment from a binomial distribution with n=1 and the estimated predictive probabilities of the relevant selected variables.

For a selected cohort having Parkinson's disease, the result of the algorithm shows an overall accuracy of 80% (76%-83%) on a test data set, which means an acceptable discrimination with new test-patient data. The selected relevant variables for predicting patients going on symptomatic treatment along 36 months were: PUTAMEN_IL (MRI imaging variable), APTTQT_sec (biological test), UPDRS2 (motor neuron activity), PUTAMEN_CL (MRI imaging variable), UPDRS1 (motor neuron activity). The predictive probabilities from those variables were adjusted by the disease duration and time when the response was observed, in order to obtain the final estimates for the probability of the patient going on symptomatic treatment.

For a selected cohort of 74 epilepsy patients with EEG historical data, the result of the algorithm shows an overall balance accuracy of 75% and AUC of 0.75 on a test data set, which means an acceptable discrimination with new test-patient data. The accuracy of the model increases by adding historical data to the algorithm, and thus the model can be more precise for variable selection.

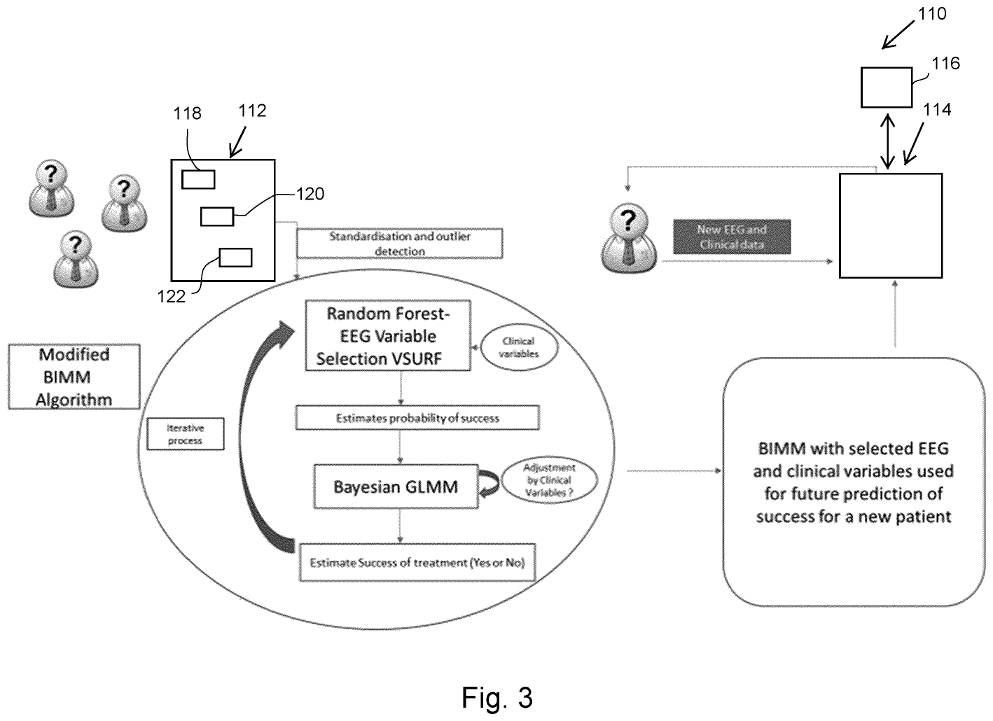

FIG. 3 shows a system 110 for predicting a response variable for a neurological condition in accordance with an example of the present disclosure. System 110 includes a central server 112 including include a memory and a processor. Server 112 may be controlled by a computer program product stored on a non-transitory computer readable media, which may be in the memory or an external storage device. The computer program product stores the machine learning algorithm trained via method 10 and may include computer executable process steps operable to control server 112 in accordance with the embodiments of method described below for predicting the response variable for the neurological condition.

System 110 also includes at least one client computer 114 for inputting historical patient data for transferring to server 112 via a computer network. Historical data (biological, neuroimaging (EEG, MRI), clinical, etc.) from patients treated for a neurological condition, for example via an anti-epilepsy drug (AED) or via a drug for treating Parkinson's symptoms, whose treatment outcome is known is collected and pushed to central server 12 for processing for predicting a binary response variable such as for example treatment response of patients with epilepsy to a specific treatment or the need of going on symptomatic treatment in patients with Parkinson disease.

Client computer 114 may be configured for interfacing with a data interface that is configured to request electronic historical medical data for a patient from an electronic medical records database 116. Server 112 may include a variable extraction tool 118 configured for extracting relevant selected variables for predicting the response variable for the neurological condition from the electronic historical medical data. Server 112 may further include a model deployment tool 120 configured for deploying the machine learning algorithm trained by method 10 to utilize the relevant selected variables for predicting the response variable for the neurological condition. Server 112 also includes a response variable prediction generator 122 configured for running the relevant selected variables through the machine learning algorithm trained in method 10.

A method is also provided for using system 110 for predicting a response variable for a neurological condition. The method includes providing the machine learning algorithm trained by method 10, and requesting, via client computer 114, electronic historical medical data for a patient having the neurological condition from electronic medical records database 116. The relevant selected variables for predicting the response variable for the neurological condition are then extracted from the electronic historical medical data, and a prediction is generated for the response variable for the patient by running the relevant selected variables through the machine learning algorithm. The method then includes generating a display representing the prediction for the response variable for the patient on client computer 114.

It will be appreciated that the invention also applies to computer programs, particularly computer programs on or in a carrier, adapted to put the systems and the methods of the invention into practice. The present invention further provides a computer program comprising code means for performing the steps of the method described herein, wherein said computer program execution is carried on a computer. The present invention further provides a non-transitory computer-readable medium storing thereon executable instructions, that when executed by a computer, cause the computer to execute the method of the present invention. The present invention further provides a computer program comprising code means for the elements of the system disclosed herein, wherein said computer program execution is carried on a computer.

The computer program may be in the form of a source code, an object code, a code intermediate source. The program can be in a partially compiled form, or in any other form suitable for use in the implementation of the method and its variations according to the invention. Such program may have many different architectural designs. A program code implementing the functionality of the method or the system according to the invention may be sub-divided into one or more sub-routines or sub-components. Many different ways of distributing the functionality among these sub-routines exist and will be known to the skilled person. The sub-routines may be stored together in one executable file to form a self-contained program. Alternatively, one or more or all of the sub-routines may be stored in at least one external library file and linked with a main program either statically or dynamically, e.g. at run-time. The main program contains at least one call to at least one of the sub-routines. The sub-routines may also call each other.

The present invention further provides a computer program product comprising computer-executable instructions implementing the steps of the methods set forth herein or its variations as set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files.

It should be noted that the above-mentioned embodiments illustrate rather than limit the invention, and that those skilled in the art will be able to design many alternative embodiments without departing from the scope of the claims. In the claims, any reference signs placed between parentheses shall not be construed as limiting the claim.

The article “a” or “an” preceding an element does not exclude the presence of a plurality of such elements. The invention may be implemented by means of hardware comprising several distinct elements, and by means of a suitably programmed computer. In the system claim enumerating several elements, several of these elements (sub-systems) may be embodied by one and the same item of hardware. The mere fact that certain measures are recited in mutually different dependent claims does not indicate that a combination of these measures cannot be used.

All references cited herein, including patents, patent applications, papers, textbooks and the like, and the references cited therein, to the extent that they are not already, are hereby incorporated herein by reference in their entirety.

What is claimed is:

1. A computer-implemented method constructing a machine learning algorithm for predicting a response variable for a neurological condition comprising:
a) providing electronic historical medical data;
b) constructing a patient cohort from the electronic historical medical data by selecting patients having the neurological condition;
c) extracting variables for characterizing the patient cohort from the electronic historical medical data
d) selecting variables from the extracted variables using a random forest defined by an initial response variable, wherein the confounder variables are excluded from the selection process;
e) fitting a Bayesian generalized linear mixed model (GLMM) using the initial response variable and the predicted probability obtained from the random forest, wherein the logit of the probability of initial response variable yᵢₜ=1 is calculated using the equation:
$$logit ( {p}_{i t} ) = {β}_{0} + {β}_{1} * RF ( {X}_{i t} ) + {β}_{j 2} {C}_{i j} + {Z}_{i t} {b}_{i t} ,$$
where RF(Xᵢₜ) is the predicted probability from the random forest of each longitudinal observation t1, . . . , Tᵢ for cluster i=1, . . . , M,
β₀ is the coefficient for the intercept,
β₁ is the coefficient for the vector of probabilities,
βᵢₜ is a parameter from the patient random effect,
Zᵢₜ is a normal distribution of the random effect,
βⱼ₂ are the parameters estimated by the model that is adjusted for the confounder variables Cᵢⱼ from the electronic historical medical data,
pᵢₜ the probability of the initial response variable yᵢₜ to be 1, and
$$logit = ln ( {p}_{it} / ( 1 - {p}_{it} ) )$$
f) extracting a predictive probability pᵢₜ from the Bayesian GLMM for each of the selected variables,
g) determining a target response variable from the predictive probability and the initial response variable;
h) using the random forest and the Bayesian GLMM to obtain final estimated predictive probabilities based on target response variable for each of the selected variables to identify relevant selected variables; and
i) constructing the machine learning algorithm to utilize the relevant selected variables for predicting the response variable for the neurological condition.

2. The method as recited in claim 1 further comprising making the target response variable a binary value.

3. The method as recited in claim 1 wherein the confounder variables are age, gender, and the time for the longitudinal response.

4. The method as recited in claim 3 wherein the predictive probability is calculated using the equation
$$logit ( {p}_{it} ) = {β}_{0} + {β}_{1} R F ( {X}_{i t} ) + {β}_{A 2} {Age}_{i} + {β}_{G 2} {Gender}_{i} + {β}_{T 2} Time + {Z}_{i t} {b}_{i t}$$

5. The method as recited in claim 1, wherein the method is iterated through steps (d)-(g) until reaching convergence to obtain the relevant selected variables.

6. The method as recited in claim 1 wherein the machine learning algorithm is constructed to determine, from the Bayesian GLMM, the response variable from a binomial distribution with n=1 and the estimated predictive probabilities of the relevant selected variables.

7. The method as recited in claim 1 wherein the using of the random forest and the Bayesian GLMM to obtain final estimated predictive probabilities based on target response variable for each of the selected variables to identify relevant selected variables includes iterating until reaching convergence:
i. determining the target response variable by adding the predictive probability extracted from the Bayesian GLMM to the initial response variable, and applying a split function to make a binary value; and then
ii. repeating the steps of selecting of the variables from the extracted variables, fitting of the Bayesian GLMM and extracting of the predictive probability using the target response variable until a change in a posterior log likelihood from the Bayesian GLMM is less than a specified tolerance value.

8. The method as recited in claim 1 wherein the selecting of variables from the extracted variables using the random forest includes:
i. computing the random forest scores of importance, then eliminating those variables with importance below a determined threshold and ordering the remaining variables in decreasing order of importance; and
ii. selecting the variables involving the smallest out-of-bag error from the random forest.

9. The method as recited in claim 1 wherein the response variable is whether or not a treatment for a neurological condition is expected to be successful.

10. The method as recited in claim 9 wherein the neurological condition is Parkinson's disease and the extracted variables for characterizing the patient cohort include biological variables, neuroimaging variables and clinical variables.

11. The method as recited in claim 10 wherein the selected relevant variables are PUTAMEN_IL, APTT-QT(sec), UPDRS2, PUTAMEN_CL and UPDRS1.

12. The method as recited in claim 9 wherein the neurological condition is epilepsy and the extracted variables for characterizing the patient cohort include biological variables, neuroimaging variables, and clinical variables.

13. The method as recited in claim 12 wherein the neuroimaging variables are EEG variables including EEG connectivity zones, EEG frequency bands and EEG measures.

14. A computer system for predicting a response variable for a neurological condition comprising:
1) a client configured for interfacing with a data interface server, the data interface server configured to request electronic historical medical data for a patient from an electronic medical records database;
2) a variable extraction tool configured for extracting relevant selected variables for predicting the response variable for the neurological condition from the electronic historical medical data;
3) a model deployment tool configured for deploying a machine learning algorithm trained to utilize the relevant selected variables for predicting the response variable for the neurological condition;
4) a response variable prediction generator configured for running the relevant selected variables through the machine learning algorithm, the machine learning algorithm trained for utilizing a random forest and a Bayesian generalized linear mixed model (GLMM) to generate predict a response variable for a patient having the neurological condition,
wherein the machine learning algorithm is trained using the method of claim 1.

15. The computer system as recited in claim 14 wherein the confounder variables are age, gender, and the time for the longitudinal response.

16. The computer system as recited in claim 15 wherein the predictive probability is calculated using the equation
$$logit ( {p}_{it} ) = {β}_{0} + {β}_{1} R F ( {X}_{it} ) + {β}_{A 2} {Age}_{i} + {β}_{G 2} {Gender}_{i} + {β}_{T 2} Time + {Z}_{i t} {b}_{i t}$$

17. The computer system as recited in claim 14, wherein the machine learning algorithm training method is iterated through steps (d)-(g) until reaching convergence to obtain the relevant selected variables.

18. The computer system as recited in claim 14 wherein the response variable is whether or not a treatment for a neurological condition is expected to be successful.

19. The computer system as recited in claim 14 wherein the neurological condition is Parkinson's disease and the extracted variables for characterizing the patient cohort include biological variables, neuroimaging variables and clinical variables.

20. The computer system as recited in claim 19 wherein the selected relevant variables are PUTAMEN_IL, APTT-QT(sec), UPDRS2, PUTAMEN_CL and UPDRS1.

21. The computer system as recited in claim 14 wherein the neurological condition is epilepsy and the extracted variables for characterizing the patient cohort include biological variables, electrophysiological variables and clinical variables.

22. The computer system as recited in claim 21 wherein the neuroimaging variables are EEG variables including EEG connectivity zones, EEG frequency bands and EEG measures.

23. A computerized method for predicting a response variable for a neurological condition comprising:
1) providing a machine learning algorithm trained to utilize the relevant selected variables for predicting the response variable for the neurological condition;
2) requesting, via a client, electronic historical medical data for a patient having the neurological condition from an electronic medical records database;
3) extracting relevant selected variables for predicting the response variable for the neurological condition from the electronic historical medical data;
4) generating a prediction for the response variable for the patient by running the relevant selected variables through the machine learning algorithm, the machine learning algorithm trained for utilizing a random forest and a Bayesian generalized linear mixed model (GLMM) to generate the predictions for the response variable; and
5) generating a display representing the prediction for the response variable for the patient,
wherein the machine learning algorithm is trained using the method of claim 1.

24. The computer system as recited in claim 23, wherein the confounder variables are age, gender, and the time for the longitudinal response.

25. The computer system as recited in claim 24 wherein the predictive probability is calculated using the equation
$$logit ( {p}_{it} ) = {β}_{0} + {β}_{1} R F ( {X}_{i t} ) + {β}_{A 2} {Age}_{i} + {β}_{G 2} {Gender}_{i} + {β}_{T 2} Time + {Z}_{i t} {b}_{i t}$$

26. The computer system as recited in claim 23, wherein the machine learning algorithm training method is iterated through steps (d)-(g) until reaching convergence to obtain the relevant selected variables.
